(19) United States
(12) Patent Application Publication
(10) Pub. No.: US 2018/0280206 A1
(43) Pub. Date: Oct. 4, 2018

(54) X-RAY DETECTABLE FABRIC AND ITS USE IN SURGICAL PATTIES AND SPONGES

(71) Applicant: Integra LifeSciences Corporation, Plainsboro, NJ (US)

(72) Inventor: Jason Karl MEDEIROS, New Bedford, MA (US)

(73) Assignee: Integra LifeSciences Corporation, Plainsboro, NJ (US)

(21) Appl. No.: 15/476,071

(22) Filed: Mar. 31, 2017

(51) Int. Cl. A61F 13/44; A61F 13/36

(52) U.S. Cl. CPC A61F 13/44; A61F 2210/0095; A61F 13/36

(57) ABSTRACT

An x-ray detectable fabric comprising: a first set of fibers having a rayon composition; a second set of fibers having a polymeric composition with an x-ray detectable material impregnated therein; wherein the first and second sets of fibers are in an entangled state as a cohesive porous fabric, and wherein fibers in the second set of fibers are distributed throughout the fabric. In particular embodiments, the first and second sets of fibers are in a non-woven state, with a bonding agent maintaining adhesion between the fibers. The polymeric composition in the second set of fibers may be selected from, for example, vinyl addition polymers, polyesters, rayon, nylon, and cellulosic compositions. The first set of fibers may or may not also include an x-ray detectable material.

### FIELD OF THE INVENTION

The present invention relates, generally, to x-ray detectable fabrics, and more particularly, to surgical patties and sponges having an x-ray detectable component incorporated therein.

### BACKGROUND OF THE INVENTION

Surgical fabrics, such as patties and sponges, are commonly used in surgical operations. In order to track the location and presence of such fabrics during an operation, or in the event such a fabric is mistakenly left in the patient after the operation, surgical fabrics commonly include an x-ray detectable component, which is typically a high x-ray absorbing element, such as barium.

In conventional practice, the x-ray detectable component in the surgical fabric is limited in its area of detection. For example, the x-ray detectable component is often applied onto a base fabric as two or more narrow bands or other pattern, e.g., U.S. Pat. No. 4,935,019. In view of the limited area of detection of the x-ray detectable component, conventional x-ray detectable fabrics have been found, in many cases, to be insufficiently detectable. Moreover, although conventional x-ray detectable fabrics may permit detection of the fabric, the full size and shape of the fabric, including the extent of incorporation into bodily tissue, remains largely unknown in view of the limited observable area of the detectable component.

Thus, there would be a significant benefit in an x-ray detectable fabric having a detectable component integrated throughout the fabric. However, until now, a viable and cost-effective manner for constructing such a unique fabric has remained elusive.

### SUMMARY OF THE INVENTION

The present invention is directed to an x-ray detectable fabric that is advantageously x-ray detectable throughout the fabric. The invention accomplishes this by entangling fibers having an x-ray detectable material impregnated therein with base fibers typically used in surgical fabrics (e.g., rayon), wherein the term “fiber” refers to a thread having sufficient flexibility so as to entangle or interweave with other threads. Although the fibers are entangled, the fabric may be a woven or non-woven fabric. The resulting fabric is characterized by the presence of x-ray detectable threading throughout the fabric.

In particular embodiments, the x-ray detectable fabric includes: a first set of fibers having a rayon composition; a second set of fibers having a polymeric composition with an x-ray detectable material impregnated therein; wherein the first and second sets of fibers are in an entangled state as a cohesive porous fabric, and wherein the fibers of the second set of fibers are distributed throughout the fabric. In some embodiments, the first set of fibers also contain x-ray detectable material impregnated therein, and may or may not be of the same composition as the second set of fibers. However, generally, a sufficient level of x-ray detectability is provided throughout the fabric with only the second set of fibers containing an x-ray detectable material (i.e., first set of fibers do not contain an x-ray detectable material), which leads to significant cost savings while at the same time providing a suitable level of x-ray detectability throughout the fabric.

The invention is also directed to methods for producing the x-ray detectable fabric described above, which may include the known methods for producing non-woven or woven fabrics, with modifications, as needed, to effectively and efficiently produce the particular types of fabric described above. For example, a non-woven version of the fabric may be produced by subjecting the two sets of fibers in an entangled state to low temperature thermo-bonding, which, as known in the art, generally employs a chemical binder to bind fabrics together. In the case of a woven version of the fabric, any of the known methods for interweaving the two sets of fibers to produce a woven fabric may be used.

### DETAILED DESCRIPTION OF THE INVENTION

In one aspect, the present invention is directed to an x-ray detectable fabric (i.e., “fabric”). The term “x-ray detectable” refers to the property of a material that is sufficiently denser than surrounding material, such as bodily tissue, to absorb more x-rays than the surrounding material, which results in the denser material having a lighter appearance than the surrounding material in a conventional x-ray negative image. Although the x-ray detectable fabric may be used for any suitable application where an x-ray detectable fabric is useful, the x-ray detectable fabric described herein is particularly considered herein for use as a surgical fabric, e.g., surgical patties and sponges. Thus, the x-ray detectable fabric may have the shape and any of the usual accessory features to make it particularly suited as a surgical patty or sponge, or more particularly, a neurosurgical patty or sponge. For example, for use as a surgical patty, the fabric may include a hanging string to facilitate location and removal of the patty after use in surgery. The x-ray detectable fabric described herein may also be used, for example, as a radiation protective article, such as a vest, gown, or drape. The fabric should be sufficiently pliable so as to follow the contours of a non-planar surface of bodily tissue when placed on bodily tissue prior to or during an operation, as generally required for a surgical fabric. The fabric is generally also rendered microbially sterile prior to use in a surgical operation. By virtue of the entanglement of fibers throughout the fabric, the fabric is cohesive (i.e., strongly resistant to unwinding) and porous. The porosity of the fabric also endows the fabric with significant liquid absorbing ability, such as the ability of the fabric to absorb at least 10, 15, or 20 times its dry weight in liquid, particularly water. Generally, the fabric considered herein has an overall thickness of up to or less than 0.5, 1, or 2 mm.

The first set of fibers has a rayon composition. In some embodiments, the first set of fibers are 100% rayon. In other embodiments, the first set of fibers is a blend of rayon fibers and one or more other types of fibers suitable for use in surgical patties or sponges. The one or more other types of fibers may be, for example, a polyester, acrylic, cellulosic (e.g., cotton), or nylon. In the case of a blend, the first set of fibers may include rayon in an amount of at least 5, 10, 20, 30, 40, 50, 60, 70, 80, 90, or 95 wt %, or within a range bounded by any of the foregoing values.

The second set of fibers has a polymeric composition with an x-ray detectable material impregnated therein. The polymeric composition is any polymeric composition known in the art that can be processed into a fiber from a melt and which is suitable for use as a surgical fabric, particularly a surgical patty or sponge. Thus, the polymeric composition should result in a fiber that is sufficiently flexible such that the resulting fabric, as a whole, retains sufficient pliability so as to follow the contours of a non-planar surface of bodily tissue when placed on bodily tissue prior to or during an operation. Generally, the second set of fibers have physical characteristics that are the same or similar to the physical characteristics of the first set of fibers. The polymeric composition of the second set of fibers can be, for example, a vinyl addition composition, polyester, rayon, nylon, or cellulosic composition. In the case of a vinyl addition composition, the vinyl addition composition may be, for example, a polyvinyl chloride, polypropylene, polyethylene, vinyl acetate, polyacrylate, or a blend or copolymer thereof. In some embodiments, the first and second sets of fibers have a rayon composition, which may be the same or different rayon compositions. In another embodiment, the first and second sets of fibers have the same rayon composition. In other embodiments, the second set of fibers has a composition that does not include rayon.

The x-ray detectable material includes at least one element commonly used in medical x-ray imaging for its ability to contrast with surrounding bodily tissue. As well known in the art, the x-ray detectable element is generally heavier than the common elements found in bodily tissue, such as carbon, calcium, nitrogen, and oxygen. Generally, the x-ray detectable element has an atomic number of at least 24, 29, 50, or 56 to provide sufficient contrast. Some examples of x-ray detectable elements include barium, iodine, tungsten, bismuth, antimony, copper, lanthanides, and actinides. The x-ray detectable element is generally in the form of a salt, such as barium sulfate. The x-ray detectable element or salt thereof is generally included in the second set of fibers in an amount of at least 5 or 10 wt % relative to the total weight of the impregnated fiber. In different embodiments, the x-ray detectable element or salt thereof is included in the second set of fibers in an amount of 5, 10, 15, 20, 25, 30, 35, 40, 45, 50, 55, 60, 65, 70, 75, 80, 85, or 90 wt % by total weight of the impregnated fiber, or in an amount within a range bounded by any two of the foregoing values (e.g., 15-90 wt %, 20-85 wt %, 40-80 wt %, or 40-70 wt %). The first set of fibers may or may not also include an x-ray detectable material. In the event the first set of fibers are also impregnated with x-ray detectable material, the x-ray detectable material may also be included in any of the exemplary amounts provided above.

In some embodiments, the second set of fibers also include an incorporated dye to render the second set of fibers visibly distinguished from the first set of fibers. The term “visibly distinguished,” as used herein, refers to the ability of the human eye to distinguish between the first and second fibers by direct visual observation under ordinary light or a particular wavelength (e.g., ultraviolet or lower energy electromagnetic radiation) under which the dye fluoresces. To ensure the visible distinction, in some embodiments, the first set of fibers are not dyed while the second set of fibers are dyed to provide the visual contrast from the undyed set of second fibers. In an alternative embodiment, the first and second sets of fibers are dyed with a first and second dye, respectively, where the first and second dyes are visually distinguishable from each other.

To produce the second set of fibers, any of the methods known in the art for producing fibers by melt extrusion of a polymeric composition can be employed herein. For example, at least one polymer and an x-ray detectable material can be combined, melted, and mixed to provide a homogeneous melt. The homogeneous melt can then be extruded, or more specifically, melt-spun, such as by processing through a spinneret with multiple extrusion ports, such as described in U.S. Application Pub. No. 2009/0000007, the contents of which are herein incorporated by reference. Alternatively, fibers of a polymeric composition can first be produced without an x-ray detectable material incorporated therein, followed by immersion of the fiber into a solution containing the x-ray detectable material in such manner that the x-ray detectable material becomes impregnated (i.e., incorporated into) the fibers.

As indicated above, the first and second sets of fibers are entangled with each other in the fabric. Generally, the fibers in the first set of fibers are also entangled with each other, and the fiber in the second set of fibers are also entangled with each other. The term “entangled,” as used herein, refers to a state in which a fiber passes multiple times through spaces delineated by one or more other fibers. Particularly in view of the cost limitations involved in weaving, the type of entanglement generally considered herein is a non-woven type of entanglement. In the non-woven state, the first and second sets of fibers are entangled in the absence of an entanglement pattern that normally arises from weaving, i.e., the first and second sets of fibers are randomly entangled. The non-woven fabrics may be prepared by, for example, conventional hydraulic entanglement methods, as described, for example, in U.S. Pat. No. 4,935,019 and references cited therein, all of which are herein incorporated by reference in their entirety. In brief, the conventional hydraulic entanglement method generally involves positioning a fibrous web of randomly oriented fibers on an apertured belt, which may or may not be patterned, and subjecting the web to a plurality of high pressure hydraulic jets to entangle the fibers. The entangled fibers are then separated from the belt and optionally dried on heated drums to produce the nonwoven fabric. The end result is that the fibers in the second set of fibers, which have the x-ray detectable material impregnated therein, are distributed throughout and within the fabric. Thus, the entire fabric, including surface and interior portions, are x-ray detectable.

Generally, to maintain bonding (i.e., adhesion or cohesion) between the entangled fibers during use, the fibers in the fabric are bonded with each other with a bonding agent (binder). Any of the well-known bonding agents may be used, such as an acrylic-based binder. The presence of the binder also advantageously aids in the reduction of linting during use of the fabric.

While there have been shown and described what are at present considered the preferred embodiments of the invention, those skilled in the art may make various changes and modifications which remain within the scope of the invention defined by the appended claims.

What is claimed is:

1. An x-ray detectable fabric comprising:
a first set of fibers having a rayon composition;
a second set of fibers having a polymeric composition with an x-ray detectable material impregnated therein;
wherein said first and second sets of fibers are in an entangled state as a cohesive porous fabric, and wherein fibers in said second set of fibers are distributed throughout the fabric.

2. The fabric of claim 1, wherein said first and second sets of fibers are in a non-woven state, with a bonding agent maintaining adhesion between the fibers.

3. The fabric of claim 1, wherein said polymeric composition is selected from vinyl addition polymers, polyesters, rayon, nylon, and cellulosic compositions.

4. The fabric of claim 3, wherein said polymeric composition is a vinyl addition polymer.

5. The fabric of claim 4, wherein said vinyl addition polymer is selected from a polyvinyl chloride, polypropylene, polyethylene, vinyl acetate, polyacrylate, and copolymers and blends thereof.

6. The fabric of claim 4, wherein said vinyl addition polymer is polypropylene.

7. The fabric of claim 1, wherein said x-ray detectable material comprises an x-ray detectable element having an atomic number of at least 24.

8. The fabric of claim 1, wherein said x-ray detectable material comprises an x-ray detectable element having an atomic number of at least 50.

9. The fabric of claim 1, wherein said x-ray detectable material comprises an x-ray detectable element selected from barium, iodine, tungsten, bismuth, antimony, copper, lanthanides, and actinides.

10. The fabric of claim 1, wherein said x-ray detectable material comprises barium as an x-ray detectable element.

11. The fabric of claim 1, wherein said x-ray detectable material is included in said second set of fibers in an amount of at least 15 wt % and up to 90 wt % relative to the total weight of the second set of fibers.

12. The fabric of claim 1, wherein said x-ray detectable material is included in said second set of fibers in an amount of at least 40 wt % and up to 80 wt % relative to the total weight of the second set of fibers.

13. The fabric of claim 1, wherein said second set of fibers further comprises an incorporated dye to render the second set of fibers visibly distinguished from the first set of fibers.

14. The fabric of claim 1, wherein said fabric has a thickness of less than 1 mm.

15. The fabric of claim 1, wherein said fabric is sufficiently pliable so as to follow the contours of a non-planar surface of bodily tissue.

16. The fabric of claim 1, wherein said fabric further comprises a hanging string to facilitate location and removal of the fabric after use in surgery, wherein the hanging string renders the fabric suited as a surgical patty.

17. The fabric of claim 1, wherein said fabric is microbially sterile.

18. The fabric of claim 1, wherein said fabric can absorb at least 10 times its dry weight in liquid.

19. The fabric of claim 1, wherein said first set of fibers do not include an x-ray detectable material.

20. The fabric of claim 1, wherein said first set of fibers include an x-ray detectable material impregnated therein.
